(19) United States
(12) Patent Application Publication
(10) Pub. No.: US 2025/0032179 A1
(43) Pub. Date: Jan. 30, 2025

(54) CATHETER FOR LACERATION OF VALVE LEAFLET AND ASSOCIATED SYSTEMS AND METHODS

(71) Applicant: Kar Health, LLC, Irvine, CA (US)

(72) Inventors: Wassef Karrowni, Irvine, CA (US); Mirna Karouni, Irvine, CA (US)

(73) Assignee: Kar Health, LLC, Irvine, CA (US)

(21) Appl. No.: 18/787,805

(22) Filed: Jul. 29, 2024

Related U.S. Application Data
(60) Provisional application No. 63/529,420, filed on Jul. 28, 2023.

(51) Int. Cl. A61B 18/14; A61B 18/00; A61M 25/00

(52) U.S. Cl. CPC A61B 18/1492; A61M 25/0041; A61B 2018/00166; A61B 2018/0022; A61B 2018/00369; A61B 2018/00601; A61B 2018/144; A61M 2025/0042

(57) ABSTRACT

A multi-lumen catheter is comprising a first lumen configured to deliver targeted and controlled puncture to the leaflet using an electrified wire and a second lumen configured to deliver a loop-snare, that captures the electrified wire after the electrified wire punctures the leaflet, wherein the first lumen and the second lumen are in close proximity and same spatial orientation in order to facilitate the steps of puncturing the leaflet with a wire and then snaring it in a timely and safe manner.

### CROSS-REFERENCE TO RELATED APPLICATION(S)

This application claims the benefit of U.S. Provisional Application No. 63/529,420 filed Jul. 28, 2023, entitled “SINGLE MULTI-LUMEN CATHETER FOR INTENTIONAL LACERATION OF VALVE LEAFLET”, which is hereby incorporated by reference in its entirety under 35 U.S.C. § 119(e).

### TECHNICAL FIELD

The disclosure relates to transcatheter aortic valve replacement (TAVR) and devices, methods, and systems for use therewith.

### BACKGROUND

Transcatheter aortic valve replacement (TAVR) is an effective and minimally invasive procedure to treat stenosis in a native aortic valve and stenosis/regurgitation in a failed bioprosthetic valve, whether a prior surgical valve or TAVR valve. TAVR for treatment of a failed bioprosthetic valve, also known as valve-in-valve (VIV) TAVR, carries an increased risk of occlusion of one or both coronary ostia and a risk of sequestration or effacement of the coronary sinuses. These risks also exist for TAVR for treatment of native aortic stenosis, though not as frequently. As would be understood, an implanted TAVR valve pins open the leaflets of the first existing valve which, in certain cases, may push the leaflets into the origin of the coronary artery or may create a tube graft or covered stent within the ascending aorta, which can sequester the sinuses of Valsalva and obstruct blood flow to the coronary arteries.

Computed tomography (CT) scans are routinely performed prior to a TAVR procedure in order to study the anatomy of the existing native aortic valve or bioprosthetic valve and to decide on the size of the TAVR bioprosthetic valve that needs to be implanted and also to evaluate potential procedure related complications. Anatomical variables to study in these scans prior to a TAVR procedure include the distance of the coronary arteries origin from the aortic valve annulus, the length and bulkiness of valve leaflets, the flaps that open and close, and the height of the sinotubular junction. This evaluation is done in order to avoid occlusion of the coronary artery when the new valve's scaffold pushes the existing leaflets into the coronary arteries or sequestration of the sinuses of Valsalva.

BASILICA (Bioprosthetic or Native Aortic Scallop Intentional Laceration to Prevent Latrogenic Coronary Artery Obstruction) has emerged as a technique to prevent coronary artery obstruction or sequestration of the sinuses of Valsalva in patients at risk during TAVR. BASILICA uses transcatheter electrosurgery to lacerate or split the aortic leaflets before TAVR allowing them to splay to the sides after TAVR and, thus allows for flow of blood through the lacerated leaflet into the sinus of Valsalva and coronary artery ostium. The BASILICA technique has several limitations including that it is technically challenging, requires a high level of expertise, and involves the simultaneous and coordinated use of two separate catheter systems which increases the complexity and time spent doing the procedure.

The first step of a BASILICA procedure is to use guiding catheters and an electrified wire in order to puncture and traverse the leaflet of interest at its center and base. This step is technically challenging and time consuming. This step also risks the wire inadvertently puncture neighboring structures leading to complications. The next step in the BASILICA technique is to capture the wire that pierced the leaflet via loop-snare, introduced by a separate catheter and precisely positioned in the left ventricular outflow tract (LVOT). This step requires meticulous coordination between the movements of the formerly mentioned two catheters (which often requires two surgeons/operators) and thus adds to the complexity, cost, and availability of the procedure.

### BRIEF SUMMARY

Disclosed herein are devices, systems, and methods that allow for precise and safe puncture and traversal of a valve leaflet. The disclosed devices, systems, and methods include dedicated catheters to accomplish the steps of the BASILICA technique in a more coordinated and timely manner.

In one Example a multi-lumen catheter is comprising: a first lumen configured to deliver targeted and controlled puncture to the leaflet using an electrified wire; a second lumen configured to deliver a loop-snare, that captures the electrified wire after the electrified wire punctures the leaflet, wherein the first lumen and the second lumen are in close proximity and same spatial orientation in order to facilitate the steps of puncturing the leaflet with a wire and then snaring the wire.

In Example 1, a multi-lumen catheter is comprising a first lumen configured to deliver targeted and controlled puncture to a leaflet using an electrified wire and a second lumen configured to deliver a loop-snare, wherein the loop-snare is configured to capture the electrified wire after the electrified wire punctures the leaflet, and wherein the first lumen and the second lumen are in close proximity and same spatial orientation.

In Example 2, a catheter comprising a first lumen configured for insertion of a puncturing wire and a second lumen configured for insertion of a loop-snare, wherein the puncturing wire is configured to puncture a valve leaflet and be captured by the loop-snare.

Example 3 relates to the catheter of any of Examples 1-2 and 4-14, further comprising a third lumen configured for insertion of a guide wire.

Example 4 relates to the catheter of any of Examples 1-3 and 5-14, wherein the catheter is steerable.

Example 5 relates to the catheter of any of Examples 1-4 and 6-14, further comprising a microcatheter disposed within the first lumen.

Example 6 relates to the catheter of any of Examples 1-5 and 7-14, wherein the puncturing wire comprises a marker at a distal end of the puncturing wire for visualization during a procedure.

Example 7 relates to the catheter of any of Examples 1-6 and 8-14, wherein the leaflet is an aortic valve leaflet.

Example 8 relates to the catheter of any of Examples 1-7 and 9-14, further comprising a needle at a distal end of the puncturing wire.

Example 9 relates to the catheter of any of Examples 1-8 and 10-14, wherein the second lumen distal end is above the first lumen distal end.

Example 10 relates to the catheter of any of Examples 1-9 and 11-14, further comprising a balloon at a distal end of the catheter.

Example 11 relates to the catheter of any of Examples 1-10 and 12-14, further comprising a pigtail extension disposed on the distal end of the first lumen

Example 12 relates to the catheter of any of Examples 1-11 and 13-14, wherein the pigtail extension comprises a marker for visualization during a procedure.

Example 13 relates to the catheter of any of Examples 1-12 and 14, wherein the first lumen of the catheter ends at the base of pigtail extension; the pigtail extension shaped and arranged to support directing the puncture of the leaflet away from a free edge of the leaflet.

Example 14 relates to the catheter of any of Examples 1-13, wherein the pigtail extension and the second lumen have similar orientation.

In Example 15, a method of splitting a valve leaflet, comprising inserting a single catheter to a target location in a body of a patient; inserting a puncturing wire into a first lumen of the single catheter and advancing the puncturing wire through the first lumen; electrifying the puncturing wire and advancing the electrified puncturing wire through the valve leaflet; advancing a microcatheter over the puncturing wire; inserting a wire through the microcatheter and into the body of the patient where the wire can float through the valve orifice; snaring the wire with a loop snare inserted into a second lumen of the single catheter; externalizing the wire through the second lumen; and lacerating the valve leaflet.

Example 16 relates to the method of any of Examples 15 and 17-20, further comprising rotating the single catheter to be in position against the valve leaflet.

Example 17 relates to the method of any of Examples 15-16 and 18-20, further comprising inflating a balloon when the single catheter reaches the target location.

Example 18 relates to the method of any of Examples 15-17 and 19-20, further comprising inserting a guide wire into the body of the patient through a third lumen.

Example 19 relates to the method of any of Examples 15-18 and 20, further comprising sequentially performing the steps of Example 15 for splitting multiple valve leaflets of a valve in a single procedure.

Example 20 relates to the method of any of Examples 15-19, wherein the valve leaflet is a leaflet of the aortic valve.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a schematic view of the catheter in place in a valve, according to one implementation.

FIG. 2 is a schematic view of the catheter of FIG. 1 in place in the valve with a wire advanced through a first lumen and a loop-snare advanced through a second lumen, according to one implementation.

FIG. 3 is a schematic view of the catheter of FIGS. 1-2 with the wire in contact with the leaflet tissue and advancing into the left ventricle, according to one implementation.

FIG. 4 is a schematic view of the catheter of FIGS. 1-3 where the wire and a microcatheter has advanced through an opening in the leaflet, according to one implementation.

FIG. 5 is a schematic view of the catheter of FIGS. 1-4 where the microcatheter has advanced over the wire and a soft tipped wire is floated into the ascending aorta, according to one implementation.

FIG. 6 is a schematic view of the catheter of FIGS. 1-5 where the soft tipped wire has been captured by a loop snare, according to one implementation.

FIG. 7 is a schematic view of a catheter with a balloon expanded at a distal end of the catheter, according to one implementation.

FIG. 8 is a schematic view of a catheter having three lumens, according to one implementation.

FIG. 9 is a schematic view of the catheter of FIG. 8 with a guide wire in a first lumen and a loop snare advanced through a second lumen, according to one implementation.

FIG. 10 is a schematic view of the catheter of FIGS. 8-9 with an over-the-wire microcatheter advanced through a third lumen, according to one implementation.

FIG. 11 is a schematic view of the catheter of FIGS. 8-10 where a puncturing wire is advanced through the microcatheter to contact a valve leaflet, according to one implementation.

FIG. 12 is a schematic view of the catheter of FIGS. 8-11 where the puncturing wire is electrified and punctures the leaflet and is thereafter captured by the loop snare, according to one implementation.

FIG. 13 is a schematic view of the catheter of FIGS. 8-12 where the loop snare and wire are moved through the second lumen to the outside of the body thereby lacerating the leaflet, according to one implementation.

### DETAILED DESCRIPTION

Described herein are various devices, systems, and methods for use in performing a BASILICA technique during or prior to a TAVR procedure or alternative similar procedures. More specifically provided herein are catheters capable of easy, controlled execution of splitting the valve leaflets, including the aortic valve before TAVR thereby allowing the leaflets to splay to the sides after TAVR thereby avoiding certain TAVR complications. It would be understood that the described catheters and procedures may be used with any valve. Various alternative uses for the herein disclosed catheters and associated methods and systems are possible, would be recognized by those of skill in the art, and are contemplated herein.

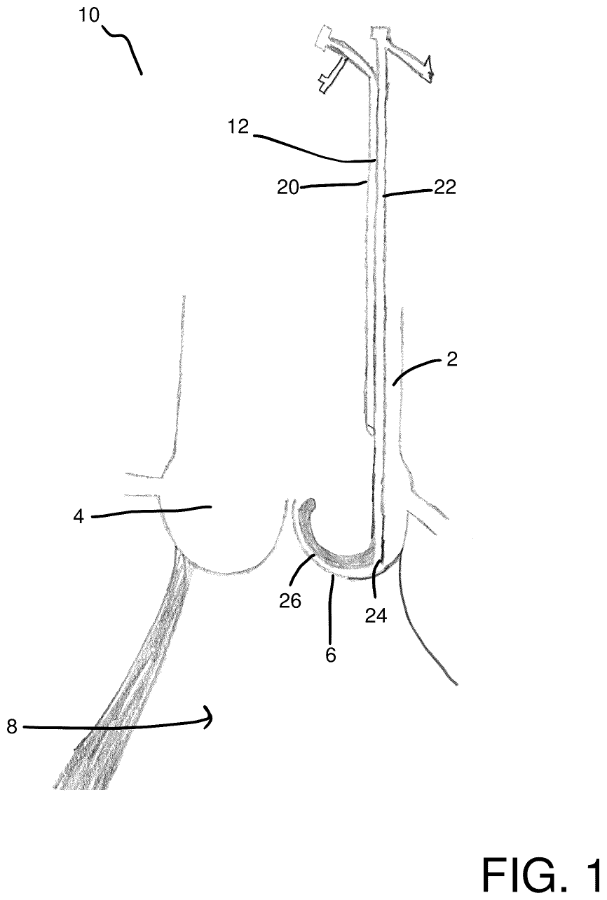

Turning to the figures, FIG. 1 shows one exemplary implementation of a dual lumen catheter 10. In various implementations, the catheter 10 includes an elongated flexible shaft 12 that is configured to be inserted into the body of a patient. Insertion of the catheter 10 into the body can be done by any clinically appropriate technique, as would be understood. For example, the catheter 10 may be inserted through a sheath into an artery using standard techniques.

In various implementations, the catheter 10 is steerable and may optionally have pressure transducer. Various additional features may be present on/with the catheter 10 as would be understood. In various implementations, the catheter 10 shaft comprises a flexible shaft material, such as woven Dacron or synthetic materials such as polyurethane or others, as would be appreciated.

As can be seen in FIG. 1, in certain implementations, the distal end of the catheter 10 has a pigtail-shape 26. This pigtail end/part 26 may be a flexible component that may be extended straight (for example in order to introduce the part 26 inside the body through a sheath) but once released would take its memory shape of pigtail 26. The pigtail shape 26 may optionally include radiopaque markers to facilitate visualization during procedure. As would be understood by those in the art, “distal” refers to the portion of an object that is furthest from the user, such as a surgeon. Similarly, and as would also be understood, “proximal” refers to the portion of an object that is closest from the user, such as a surgeon.

As would be understood, the pigtail component 26 both provides safe navigation of the catheter 10 inside the vasculature or cardiac chambers and also facilitates positioning of the distal end of the catheter 10 within a valve, optionally in a specific coronary sinus 4 of the aortic root 2, using standard techniques. The pigtail-shape 26 also optionally provides the ability to control directionality of the catheter 10 such that the catheter 10 can be rotated to position the base of the pigtail 26 in the aortic root 2 inside a coronary sinus facing the wall of the aorta 2 while the free edge faces the leaflet 6. In various implementations, the catheter 10 is advanced to ensure contact with the leaflet 6 tissue.

Continuing with FIG. 1, in various implementations, at the distal end of the catheter 10, optionally at the base of the pigtail 26, there is an opening 24 into a lumen 22 in the shaft 12 of the body of the catheter 10. Optionally, the opening 24 is at the outer curvature of the pigtail 26. The shape of the opening 24 may vary and be a pinpoint opening 24, slit-like opening 24, rectangular opening 24, or the like, as would be understood.

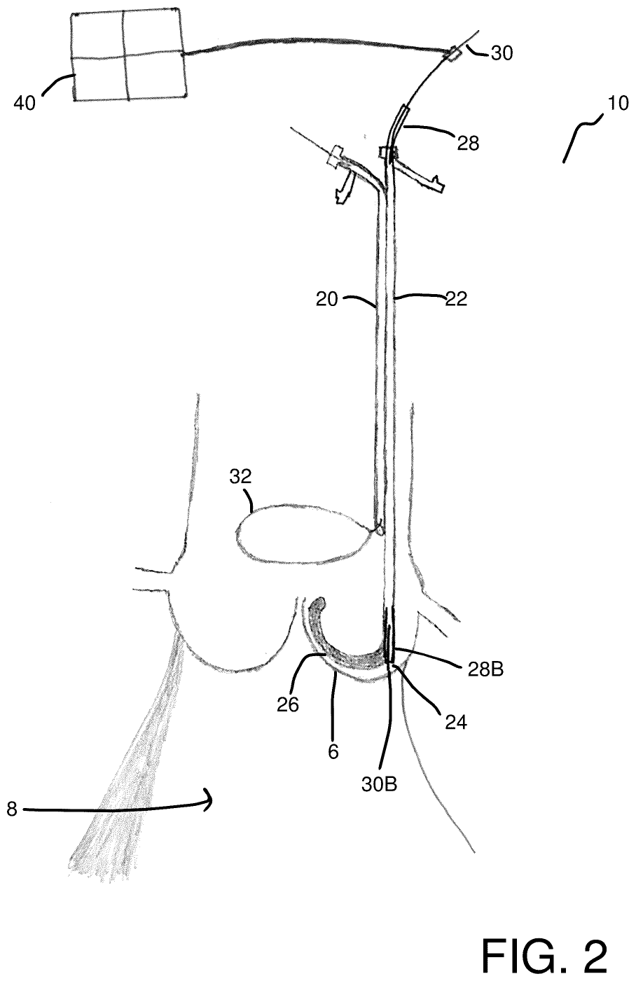

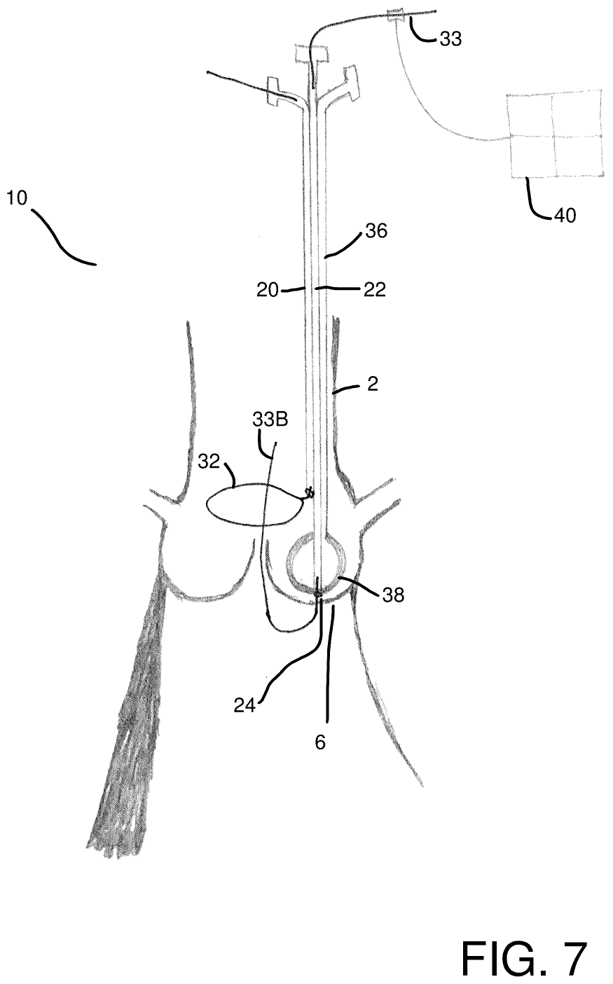

FIG. 2 shows that the lumen 22 of the catheter 10 can optionally accommodate a wire 30 and over-the-wire microcatheter 28 or over-the-wire balloon (shown in FIG. 7 and discussed further below). In various implementations, the opening 24 allows passage of a wire 30, microcatheter 28, balloon catheter, and/or a stylet into the body of the patient.

In various implementations, the puncturing wire 30 is advanced through the lumen 22 of the catheter 10 to contact the aortic valve leaflet 6 tissue at the distal end of the catheter 10. In certain implementations, the wire 30 has a radiopaque marker at its distal tip 30B. In various implementations, the proximal end of the wire 30 (base of the wire 30) is connected to an energy source 40, optionally an RF energy source 40, using standard devices and techniques.

The wire 30 may be a 0.014, 0.018, or 0.035 inch wire 30, or other appropriate size. The memory shape of the distal end 30B of the puncturing wire 30 could be: straight, angulated, J-shaped, or pigtail-shaped, such that once the wire 30 exists the lumen 22 the wire 30 assumes its memory shape.

In certain implementations, the distal end 30B of the wire 30, may optionally include a sharp point/needle that is pushed through the leaflet tissue using mechanical force by sliding a handle or similar device forward. In various implementations, the needle/wire could have an inner lumen that accommodates an additional wire.

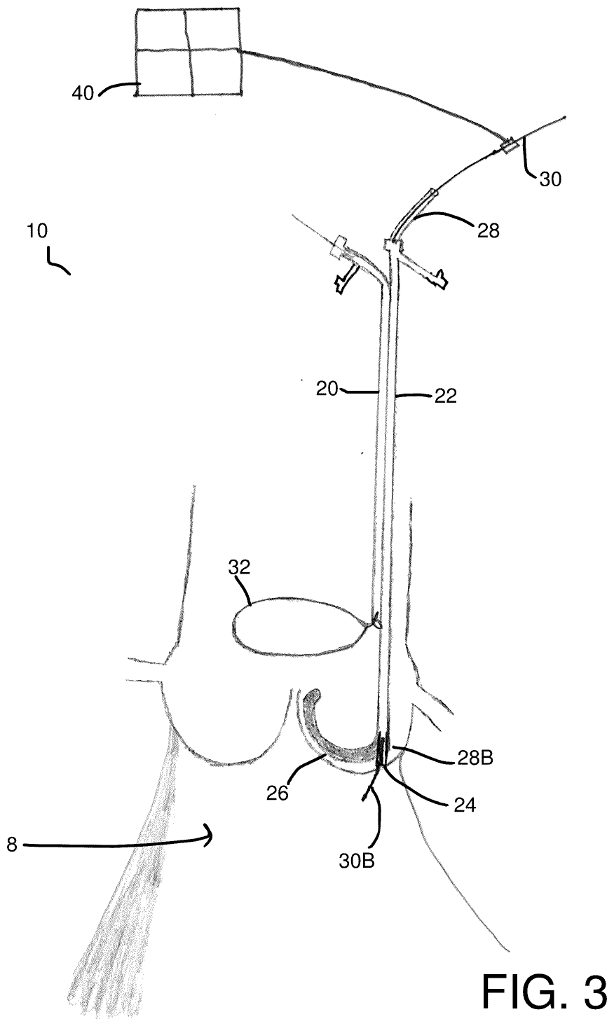

During one step of the procedure, the puncturing wire 30 is electrified while gentle forward movement is applied, advancing the distal end 30B of the wire 30 through the leaflet 6, as seen in FIG. 3. In various implementations, the wire 30 is electrified only for the time period required to lacerate the leaflet 6, this may be a short period (a second or few seconds).

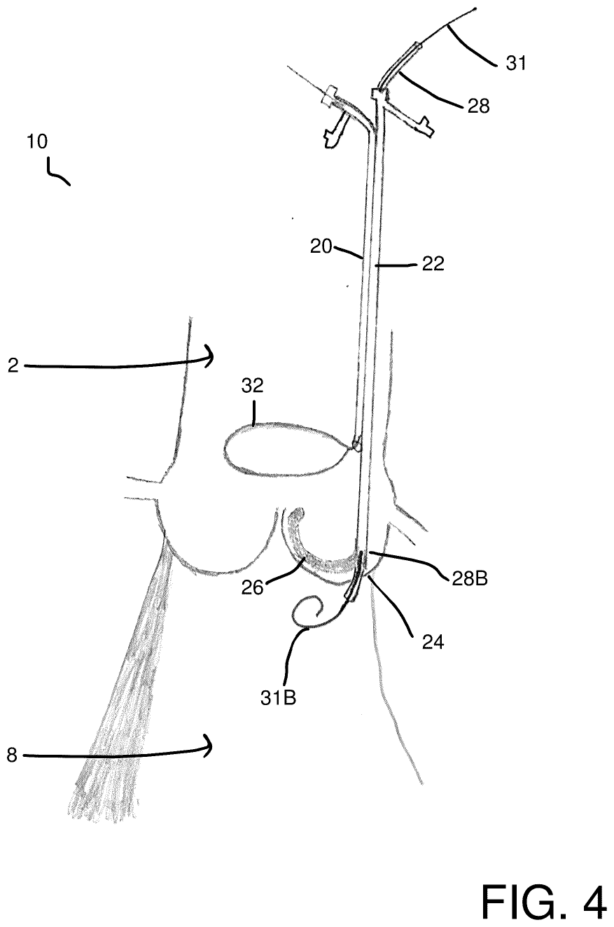

Once the leaflet 6 is punctured by the wire 30, the wire 30 can be advanced into the left ventricular outflow tract 8, optionally a centimeter or more. Once the puncturing wire 30 is advanced into the left ventricular outflow tract 8 for a specific distance, and once it has taken its memory shape (straight, angulated, J-shaped, or pigtail-shaped), in other step, a micro-catheter 28 may optionally be advanced over the wire 30 inside the lumen 22 of the catheter 10 to be positioned through the punctured aortic valve leaflet 6 and into the left ventricular outflow tract 8, as seen in FIG. 4.

Inside the microcatheter 28, and after the puncturing wire 30 is pulled out of the catheter 10, in a further step, a supportive guiding wire 31 with pigtail shaped distal end 31B may be advanced into the apex of the left ventricle 8.

The shape of the distal end 28B of the microcatheter 28 may be: straight, angulated, J-shaped, or pigtail-shaped. The supportive guiding wire 31 could be of 0.014, 0.018, or 0.035 inch caliber, or other appropriate size, as would be appreciated.

The supportive guiding wire 31 is optionally positioned in the apex of the left ventricle and may be used as a rail to advance further therapies to the leaflet 6 such as for dilating the punctured hole with a balloon of various diameters, and compliance, using standard devices and techniques. The punctured hole could be dilated using specialty balloons (for example cutting balloons) to produce more modification of the valve leaflet 6, as would be understood.

The supportive guiding wire 31 could also be exchanged over the microcatheter 28 to a soft end wire that floats with the blood flow through the aortic valve into the ascending aorta 2 where it is then e snared with a loop snare 32 to complete the known standard steps of Basilica Procedure.

In implementations, the catheter 10 may have a proximal end-to-end second lumen 20, optionally terminating just above the end of the first lumen 22 (around 1 cm from the end), shown variously and in FIG. 4. In various implementations, the second lumen 20 is oriented facing the curled portion of the pigtail 26. An opening at the distal end of the second lumen 20 is optionally located on the inner side of the catheter 10 (away from the aortic wall/toward the center of the vessel) and in parallel orientation to the pigtail 26 of the catheter 10. This lumen 26 may be used to advance a loop-snare 32 to be positioned above the valve, optionally in the proximal ascending aorta 2. The second lumen 20 could also accommodate a microcatheter 28.

In various implementations, the loop-snare 32 could be 0.014, 0.018, or 0.034 inches in caliber, or other appropriate size. The snare 32 is to be used to capture the wire 30, 31, 33 that has punctured the leaflet 6 and advanced forward through the valve to the vessel, optionally the aorta.

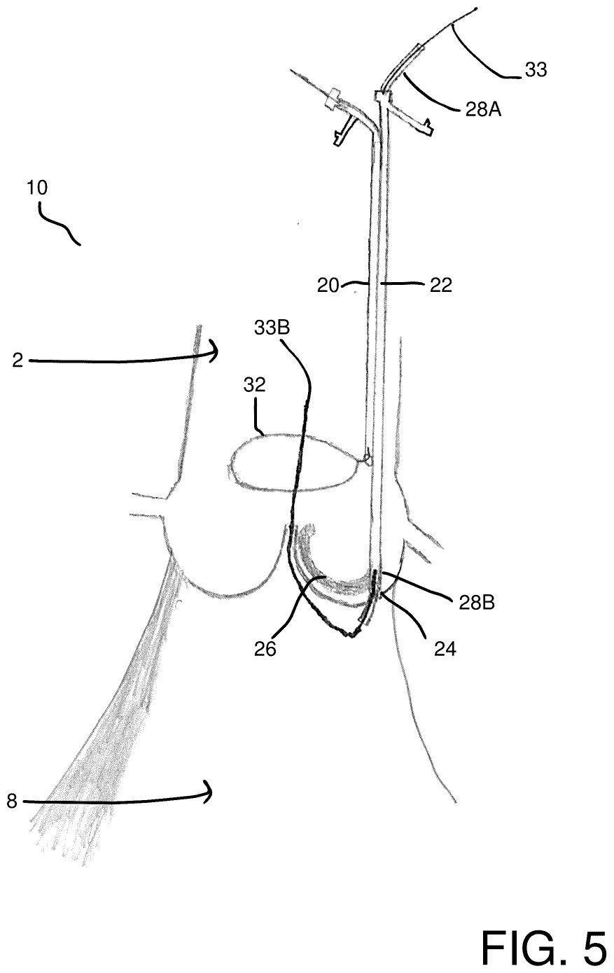

Turning to FIG. 5, after puncturing the leaflet 6 and advancing the puncturing wire 30 followed by microcatheter 28 the left ventricle, a soft ended wire 33 can be advanced through the microcatheter 28 to the left ventricle outflow tract 8 and allowed to float with the blood flow through the aortic valve orifice into the ascending aorta 2. The soft ended wire may be a standard soft ended wire 33, that would be known to those of skill in the art. The soft ended wire may be of 0.014 or 0.018 inch caliber.

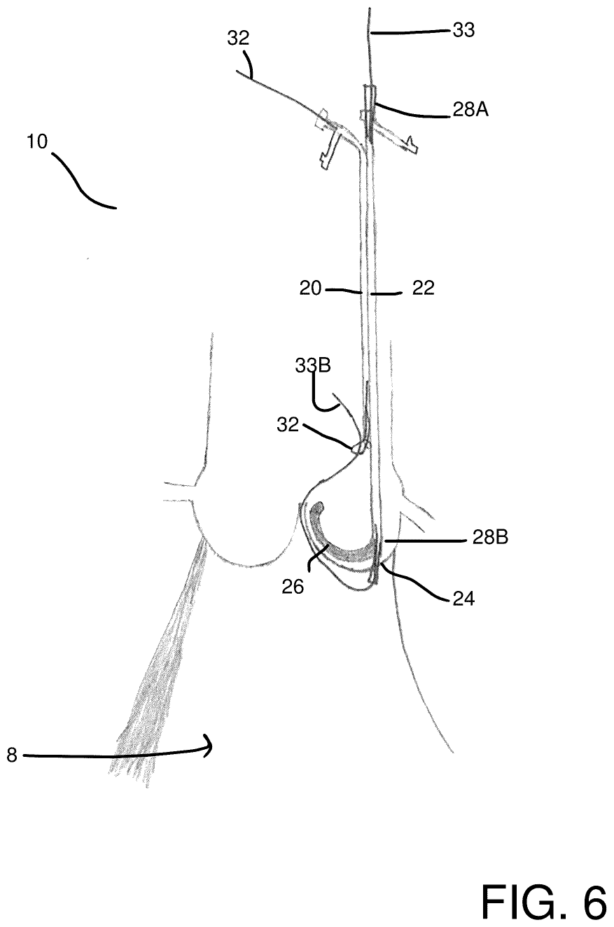

With the snare 32 advanced into the body via the lumen 20, the snare 32 can capture the floating wire 33 in the ascending aorta 2. The snare 32 may then pull-out or externalize the wire 33 through the second lumen 20 of the catheter 10, as seen in FIG. 6. This technique forms a wire 33 loop around the leaflet 6 of the aortic valve.

The leaflet 6 of the aortic valve is looped by the wire 33 that enters through a first lumen 22 of the catheter 10 and exits through a second lumen 20. With ends of the wire 33 externalized (outside the body), controlled slicing or laceration of the captured leaflet 6 using electrocautery may be executed. Various standard techniques would be known and appreciated for executing the slicing/laceration of the leaflet 6.

Turning to FIG. 7, in various implementations, the catheter 10 may include a third lumen 36 for insertion of an inflatable balloon 38 or other expandable member 38 at its distal end. In these and other implementations, the balloon 38 is used in place of the pigtail 26, discussed above.

In various implementations, the catheter 10 first lumen 22 may be inside the expandable member 38 and end with the opening 24 at the tip of the expandable member 38.

As would be understood, the balloon 38 may be compliant, semi-complaint, or non-compliant. The shape of the balloon 38 or expandable part 38 could be round, oval, semilunar, or any other shape. The balloon 38 can be filled with contrast, saline, or other fluid mixture. The size of the balloon 38 could vary per patient anatomy, but common sizes are 3 mm to 10 mm. Inflation and deflation of the balloon 38 can be controlled by tubing using standard techniques.

In various implementations, a balloon 38 tipped catheter 10 could be floated inside a cardiac structure or vasculature with the balloon 38 inflated, or partially inflated to avoid trauma, as would be understood.

In various implementations, the catheter 10 is advanced through a sheath inserted in an artery (commonly the common femoral artery) and advanced to the ascending aorta. This advancement could also be done over a guiding wire 31, using standard techniques, that fits inside the lumen 22 of the catheter 10. The advancement could also be done by inflation the balloon 38 once the distal end of the catheter 10 is inside an artery and floating the catheter 10 to the intended location.

The advancement of the catheter 10 may be guided by fluoroscopy and/or echocardiographic cardiac imaging. Advancement may be facilitated by manipulation of the catheter 10 by rotation and/or pull and push movements, as would be understood. Advancement could also be facilitated using a guiding sheath positioned in the ascending aorta.

Once at the level of the ascending aorta 2/aortic root 2, or other target location the balloon 38 tipped catheter 10 is advanced further (optionally with the balloon 38 inflated) into the targeted coronary sinus area 4 of the aortic valve (most commonly the left coronary sinus). The intended area is on the surface of the aortic valve facing the aortic root 2.

The expandable part 38 of the catheter 10 is expected to sit in the coronary sinus of the aortic valve in close approximation and in contact with the leaflet 6 of that coronary sinus 4 ensuring continuous contact between the expandable part 38 and the leaflet 6. This approximation limits the mobility of the leaflet 6 during the procedure.

Once the catheter 10 is in the intended position and the balloon 38 inflated, forward tension is applied at the catheter 10 to ensure contact of the opening 24 with the leaflet. The opening 24 is optionally centered in the balloon 38 area, with the aortic valve leaflet tissue 6.

The steps for the procedure approximately mimic the steps of the procedure discussed above with reference to FIGS. 1-6. That is, the procedure includes lacerating the leaflet, floating a wire up through the valve, capturing the wire with a snare, and completing the procedure, as would be generally understood by those of skill in the art.

Various further implementations may include use of a distending or expandable member other than balloon 38 at the tip of the catheter 10 to safely navigate the catheter 10 to the intended location in the coronary sinus of the aortic valve. For example, the distending or expandable member other than balloon 38 could be lattice or ball of metal, for example Nitinol or other similar material, as would be appreciated by those of skill in the art.

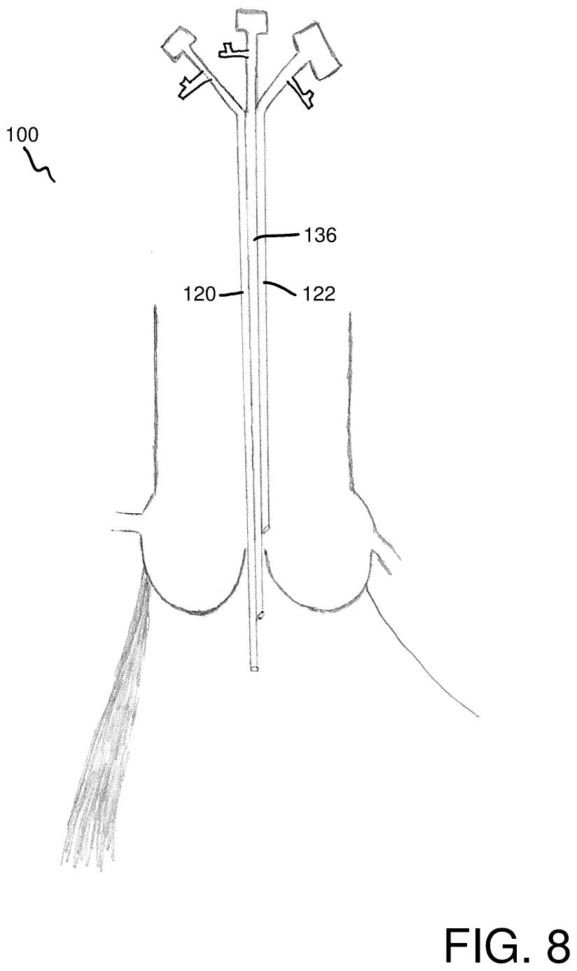

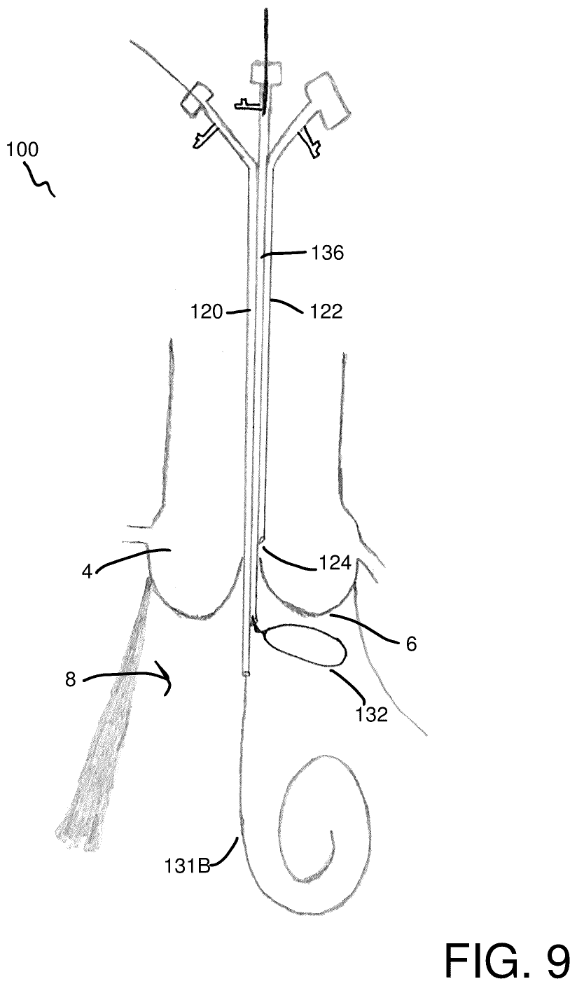

Turning now to FIGS. 8-13, in a further implementation a catheter 100 may include three lumens 120, 122, 136. In various implementations, a first lumen 120 accommodates a guide wire 131. The guide wire 131 and first lumen 120 may be extend along the entire length of the catheter 100 from the proximal end to the distal end of the catheter 100. The guide wire 131 may be used to guide catheter 100 advancement into the intended position, which is in the left ventricular outflow tract 8 through the aortic valve, as would be understood.

In various implementations, the second lumen 136 ends at the distal end of the catheter just prior (optionally about 1 cm) to the end of the first lumen 120. The end of the second lumen 136 may include an opening at its distal end where an advanced a loop-snare 132 may exit the lumen 136.

In certain implementations, the third lumen 122 ends above (more proximally than) the first lumen 120 and second lumen 136. In various implementations the third lumen 122 is on the same side of the catheter as the second lumen 136, such that the lumens 120, 136, 122 are in a lateral orientation, beside each other. The distal end of the third lumen 122 may end just above (about 2 cm) the end of the second lumen 136. In these and other implementations, the third lumen 122 is configured for advancement of a puncturing wire 130 and over-the-wire microcatheter 128.

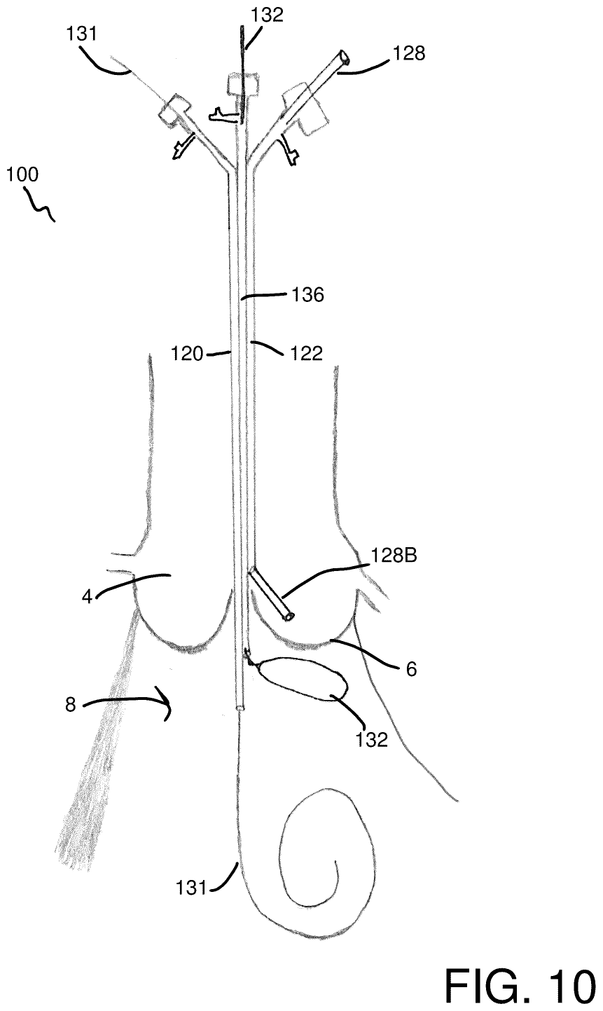

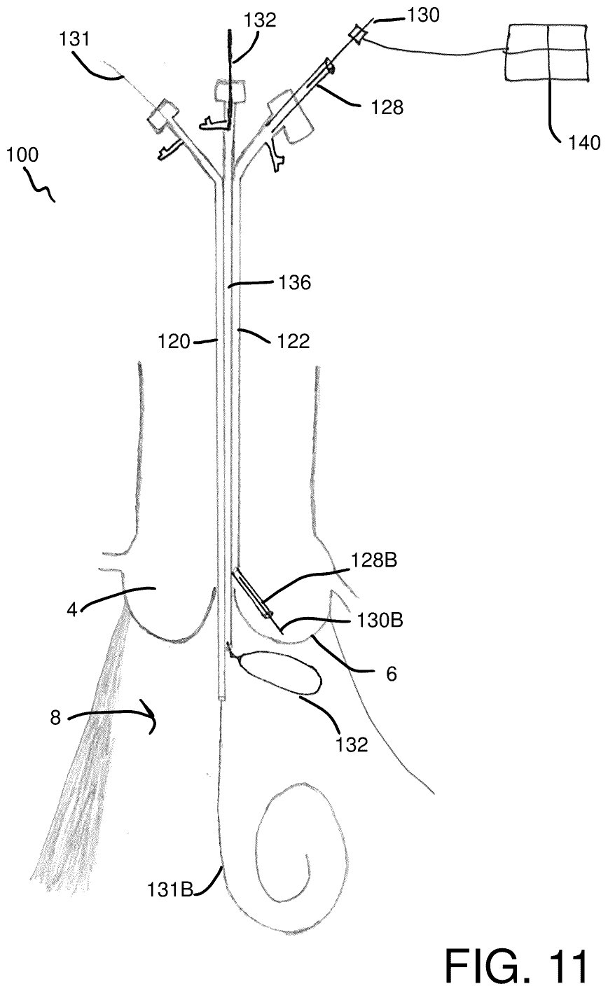

Turning to FIG. 10, the microcatheter 128 in some implementations is advanced through in the third lumen 122 and may optionally have an angled distal end 128B. For example, the distal end 128B, approximately 5-10 mm, may be angled with respect to the rest of the body of the microcatheter 128. The degree of angulation of the distal end 128B may vary for example from about 30 degrees to about 90 degrees relative to the straight shaft of the microcatheter 128. This angulation facilitates pointing the puncturing wire 130 towards the base of the valve leaflet 6, as seen in FIG. 11.

The puncturing wire 130 may have the same or similar characteristics as the puncturing wire 30 discussed above in relation to FIGS. 1-6, including a radiopaque marker at its distal tip 130B. In these and other implementations, the puncturing wire 130 is advanced inside the catheter 100 to contact the aortic valve leaflet 6 tissue at the distal end of the catheter 100.

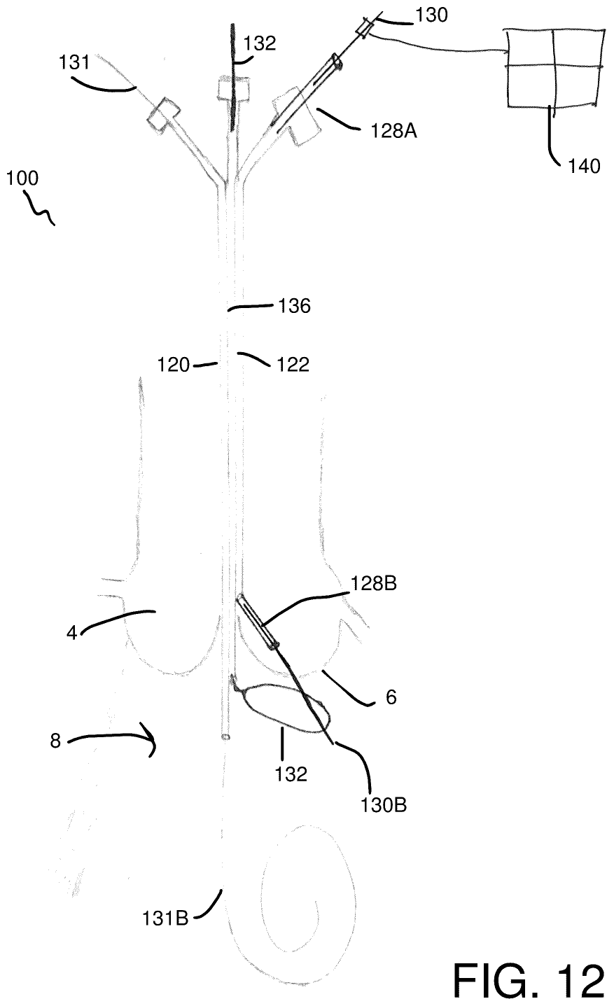

The proximal end of the puncturing wire 130 is connected to RF energy source 140 using standard techniques. As would be understood, various other methods of electrifying the puncturing wire 130 are possible, in addition to using RF. The puncturing wire 130 may be electrified for very short period (a second or few seconds) while gentle forward movement is applied. This maneuver facilitates puncturing the base of the leaflet 6 with the wire 130, which will be carefully advanced a centimeter or more into the left ventricular outflow tract 8, as seen in FIG. 12.

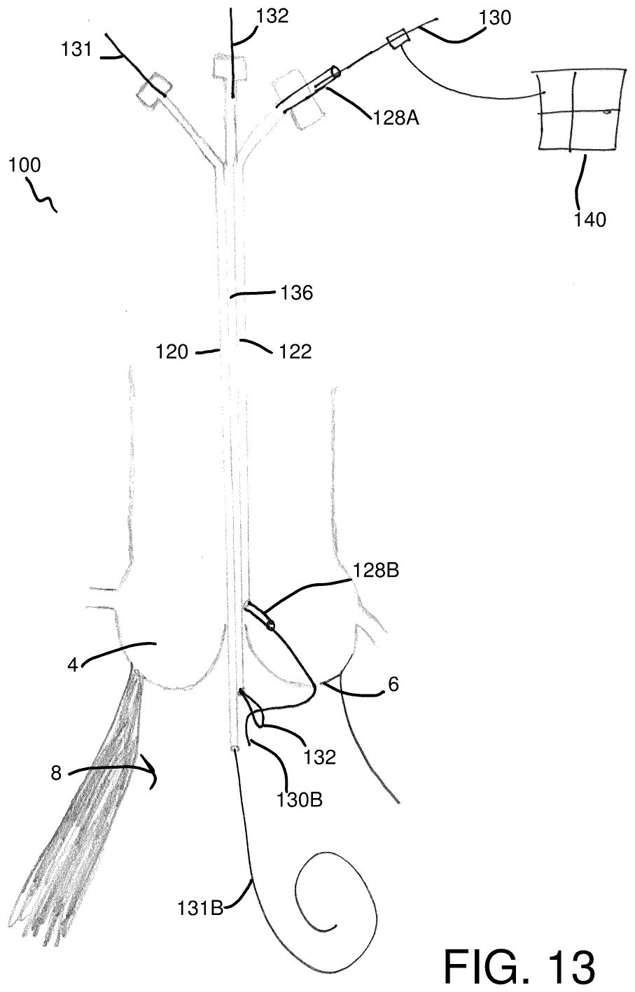

After the puncturing wire 130 is advanced into the left ventricular outflow tract 8 through the leaflet 6, it can be captured by the loop-snare 132, as seen in FIG. 13 and its distal end 130B externalized outside the body through the second lumen 136. This will create a wire 130 loop around the leaflet 6 of the aortic valve, similar to the loop discussed above with reference to FIGS. 1-6.

With the leaflet 6 of the aortic valve sandwiched by the wire 130 that enters through the third lumen 122 and exits through the second lumen 136. Both ends of the wire 130 are externalized (outside the body) to allow controlled slicing or laceration of the captured aortic leaflet 6 using electrocautery.

As would be appreciated, each of the individual sinuses of Valsalva 4 of the aortic valve can be treated sequentially as clinically needed. Alternatively, the individual leaflets of any valve many to treated sequentially with the devices and techniques provided herein. The various devices and techniques described herein are generally described in reference to us with the aortic valve and surrounding anatomy, for the sake of clarity, this is no way intended to be limiting and the various devices and techniques can be used with any valve, as would be understood by those of skill in the art.

In various implementations, the catheter shaft comprises a flexible shaft material, such as woven Dacron or synthetic materials such as polyurethane or others.

Although the disclosure has been described with references to various embodiments, persons skilled in the art will recognize that changes may be made in form and detail without departing from the spirit and scope of this disclosure.

What is claimed is:

1. A multi-lumen catheter is comprising:
(a) a first lumen configured to deliver targeted and controlled puncture to a leaflet using an electrified wire; and
(b) a second lumen configured to deliver a loop-snare,
wherein the loop-snare is configured to capture the electrified wire after the electrified wire punctures the leaflet, and wherein the first lumen and the second lumen are in close proximity and same spatial orientation.

2. A catheter comprising:
(a) a first lumen configured for insertion of a puncturing wire; and
(b) a second lumen configured for insertion of a loop-snare,
wherein the puncturing wire is configured to puncture a valve leaflet and be captured by the loop-snare.

3. The catheter of claim 2, further comprising a third lumen configured for insertion of a guide wire.

4. The catheter of claim 2, wherein the catheter is steerable.

5. The catheter of claim 2, further comprising a microcatheter disposed within the first lumen.

6. The catheter of claim 2, wherein the puncturing wire comprises a marker at a distal end of the puncturing wire for visualization during a procedure.

7. The catheter of claim 2, wherein the leaflet is an aortic valve leaflet.

8. The catheter of claim 2, further comprising a needle at a distal end of the puncturing wire.

9. The catheter of claim 2, wherein the second lumen distal end is above the first lumen distal end.

10. The catheter of claim 2, further comprising a balloon at a distal end of the catheter.

11. The catheter of claim 2, further comprising a pigtail extension disposed on the distal end of the first lumen

12. The catheter of claim 11, wherein the pigtail extension comprises a marker for visualization during a procedure.

13. The catheter of claim 11, wherein the first lumen of the catheter ends at the base of pigtail extension; the pigtail extension shaped and arranged to support directing the puncture of the leaflet away from a free edge of the leaflet.

14. The catheter of claim 11, wherein the pigtail extension and the second lumen have similar orientation.

15. A method of splitting a valve leaflet, comprising:
inserting a single catheter to a target location in a body of a patient;
inserting a puncturing wire into a first lumen of the single catheter and advancing the puncturing wire through the first lumen;
electrifying the puncturing wire and advancing the electrified puncturing wire through the valve leaflet;
advancing a microcatheter over the puncturing wire;
inserting a wire through the microcatheter and into the body of the patient where the wire can float through the valve orifice;
snaring the wire with a loop snare inserted into a second lumen of the single catheter;
externalizing the wire through the second lumen; and
lacerating the valve leaflet.

16. The method of claim 15, further comprising rotating the single catheter to be in position against the valve leaflet.

17. The method of claim 15, further comprising inflating a balloon when the single catheter reaches the target location.

18. The method of claim 15, further comprising inserting a guide wire into the body of the patient through a third lumen.

19. The method of claim 15, further comprising sequentially performing the steps of claim 15 for splitting multiple valve leaflets of a valve in a single procedure.

20. The method of claim 15, wherein the valve leaflet is a leaflet of the aortic valve.
